(19) United States
(12) Patent Application Publication
(10) Pub. No.: US 2025/0223255 A1
(43) Pub. Date: Jul. 10, 2025

(54) METHOD FOR PRODUCING ANILINE OR AN ANILINE-DERIVED PRODUCT

(71) Applicant: Covestro Deutschland AG, Leverkusen (DE)

(72) Inventors: Martin Machat, Köln (DE); Sandy Lama, Wiener Neustadt (AT); José Fonseca, Vigo (ES); Eric Ritter, Köln (DE); Volker Michele, Köln (DE); Annika Anheyer, Köln (DE); Wolf Kloeckner, Köln (DE); Johannes Kiecherer, Köln (DE)

(21) Appl. No.: 18/853,466

(22) PCT Filed: Mar. 3, 2023
(86) PCT No.: PCT/EP2023/057424

§ 371 Date: Oct. 2, 2024

(30) Foreign Application Priority Data
Apr. 6, 2022 (EP) 22166914.6
Oct. 17, 2022 (EP) 22201986.1

(51) Int. Cl. C07C 209/68

(52) U.S. Cl. CPC C07C 209/68

(57) ABSTRACT

The present invention relates to a process for preparing aniline or an aniline conversion product, comprising the steps of (A) providing aminobenzoic acid; (B) decarboxylating the aminobenzoic acid in a reactor at a reaction temperature in the range from 170° C. to 350° C. to give aniline and carbon dioxide, wherein the decarboxylation is conducted at a reaction pressure at which the boiling point of aniline is reached or—preferably—exceeded, such that a first, liquid, phase possibly containing solid particles and a second, gaseous, phase form in the reactor, with a gaseous stream containing aniline and carbon dioxide being discharged from the reactor; and (C) condensing and optionally purifying the aniline present in the gaseous stream; and (D) optionally, converting the aniline obtained in (C) to an aniline conversion product.

The invention on which this application is based was financially supported by the German Federal Ministry of Food and Agriculture as part of the project “Biobasierte Herstellung von Intermediaten für Polyurethane—Phase II (Bio4PURPro)” [“Biobased Preparation of Intermediates for Polyurethanes—Phase II (Bio4PURPro)” ](funding code 22019918).

The present invention relates to a process for preparing aniline or an aniline conversion product, comprising the steps of (A) providing aminobenzoic acid; (B) decarboxylating the aminobenzoic acid in a reactor at a reaction temperature in the range from 170° C. to 350° C. to give aniline and carbon dioxide, wherein the decarboxylation is conducted at a reaction pressure at which the boiling point of aniline is reached or exceeded, such that a first, liquid, phase possibly containing solid particles and a second, gaseous, phase form in the reactor, with a gaseous stream containing aniline and carbon dioxide being discharged from the reactor; and (C) condensing and optionally purifying the aniline present in the gaseous stream; and (D) optionally, converting the aniline obtained in (C) to an aniline conversion product.

The preparation of aniline by decarboxylation of aminobenzoic acid is known in principle in the prior art. By way of example, reference may be made to the international patent applications WO 2015/124686 A1 and WO 2015/124687 A1 and the literature cited therein. The aminobenzoic acid starting compound can be obtained chemically or, preferably, fermentatively.

The chemical preparation of aminobenzoic acid is known. A suitable synthesis route is, for example, the reaction of phthalimide with sodium hypochlorite. Phthalimide can itself be obtained from phthalic anhydride and ammonia.

The fermentative preparation of aminobenzoic acid is likewise known and described in the literature; see, for example, the applications WO 2015/124686 A1 and WO 2015/124687 A1 already mentioned and the literature cited in each.

WO 2015/124686 A1 describes the decarboxylation of fermentatively or chemically prepared anthranilic acid with extraction of aniline formed in the decarboxylation with an organic solvent extraneous to the system (an alcohol, phenol, amide, ether or aromatic hydrocarbon; in particular, 1-dodecanol is emphasized as a suitable solvent).

WO 2015/124687 A1 describes the performance of the decarboxylation of fermentatively prepared anthranilic acid inter alia in water or in an organic solvent extraneous to the system, in particular 1-dodecanol, optionally in a mixture with aniline (cf. page 18, lines 28 and 29). In addition, this document also describes the option of performing the decarboxylation in aniline (without 1-dodecanol; see FIGS. 35 and 37 to 38 and the accompanying passages of text), optionally in the presence of 10% by mass of water (see FIG. 36 and the accompanying passages of text).

WO 2018/002088 A1 describes a process in which aminobenzoic acid is decarboxylated in a mixture with crude aniline. The crude aniline originates from the process itself, in that some of the product stream is not sent for purification but rather is recycled into the process. Catalysts described for the performance of the decarboxylation are aqueous acids such as sulfuric acid, nitric acid and hydrochloric acid; solid acids such as zeolites and Si—Ti molecular sieves, solid bases such as hydroxyapatites and hydrotalcites; and polymeric acids such as ion-exchange resins (in particular Amberlyst).

WO 2020/020919 A1 describes the decarboxylation of aminobenzoic acid in aniline. It is speculated that aniline itself acts as a catalyst (that is to say has autocatalytic properties). Catalysts extraneous to the system are deliberately avoided. The reaction is conducted under conditions in which aniline is liquid. In certain embodiments, a gas stream is withdrawn from the reactor in order to discharge the carbon dioxide formed as coproduct. Since minor amounts of aniline are also entrained by this gas stream, it is preferable for the entrained aniline to be selectively condensed and sent to the reaction or for workup.

The as yet unpublished patent application with the reference number EP 21177307.2 relates to a process for preparing aniline or an aniline conversion product, in which aminobenzoic acid in the presence of an inorganic heterogeneous metal oxide catalyst containing a proportion by mass of Al₂O₃, based on the total mass of the metal oxides, of 40.0% to 100%, where the proportion by mass of Al₂O₃, based on the total mass of the inorganic heterogeneous metal oxide catalyst, is 25% to 100%.

None of the processes described hitherto is completely satisfactory in respect of economics, in particular as concerns the complexity of the overall process (the actual preparation of the aniline and the workup thereof by distillation). Similarly, it is also desirable to optimize the yield of aniline (by optimizing the conversion and suppressing the by-product 2-aminobenzanilide). Accordingly, there was a need for improvements in the preparation of aniline or aniline conversion products by decarboxylation of aminobenzoic acid.

Taking account of this need, the present invention provides a process for preparing aniline or an aniline conversion product, comprising the steps of:
(A) providing aminobenzoic acid;
(B) decarboxylating the aminobenzoic acid in a reactor,
   wherein the aminobenzoic acid is introduced (i) as a solid, (ii) in molten form or (iii) dissolved or suspended in a solvent
   into the reactor and converted at a reaction temperature in the range from 170° C. to 350° C., preferably 185° C. to 300° C., particularly preferably at 190° C. to 260° C., to aniline and carbon dioxide,
   wherein the conversion is conducted at a reaction pressure at which the boiling point of aniline is reached or—preferably—exceeded, such that a first, liquid, phase possibly containing solid particles and a second, gaseous, phase form in the reactor, with a gaseous stream containing aniline and carbon dioxide being discharged from the reactor; and
(C) condensing and optionally purifying the aniline present in the gaseous stream; and
(D) optionally, converting the aniline obtained in (C) to an aniline conversion product.

Conducting the conversion at a reaction pressure at which the boiling point of aniline is reached or—preferably—exceeded, means that the reaction pressure and reaction temperature are matched to each other such that the temperature in the reactor lies above the boiling point of aniline at the pressure prevailing in the reactor or is at the least equal to the boiling point. Aniline boils at 184° C. at standard pressure. If, therefore, the conversion is conducted for example at standard pressure (1.013 bar) or a pressure that differs only slightly therefrom, a reaction temperature of 184° C. or more should be chosen. If the conversion is conducted at reduced pressure, a lower reaction temperature corresponding to the pressure reduction (but not lower than 170° C.) can also be chosen. If the conversion is conducted at elevated pressure, a higher reaction temperature corresponding to the pressure increase (but not more than 350° C.) should be chosen.

As a result, aniline formed by decarboxylation spontaneously evaporates in the process according to the invention. The aniline thus passes at least to a very predominant extent into the second, gaseous, phase and is discharged from the reactor with the gaseous stream containing aniline and carbon dioxide. It cannot be ruled out that minor proportions of the aniline formed (in particular up to 5.0%, preferably up to 2.0%, particularly preferably up to 1.0%, of the theoretical yield of aniline, with the “theoretical yield” being based on 100% conversion of the aminobenzoic acid to aniline, where 1 mol of aminobenzoic acid is converted to 1 mol of aniline) remain in the first, liquid, phase possibly containing solid particles and are then for example discharged with a purge stream (see further below for details) and disposed of. This does not constitute a departure from the scope of the present invention.

Surprisingly, it has been found that conducting the decarboxylation under conditions in which the aniline formed passes into the gas phase and is drawn off in gaseous form represents a simple configuration option for the process that has the feature of a high conversion and high selectivity for the target product aniline. In the process according to the invention, the “crude aniline” that comes from the reaction is effectively already distilled, which considerably simplifies further purification, if this is necessary at all.

The appended drawings show reactors that are suitable for the continuous performance of the process according to the invention:

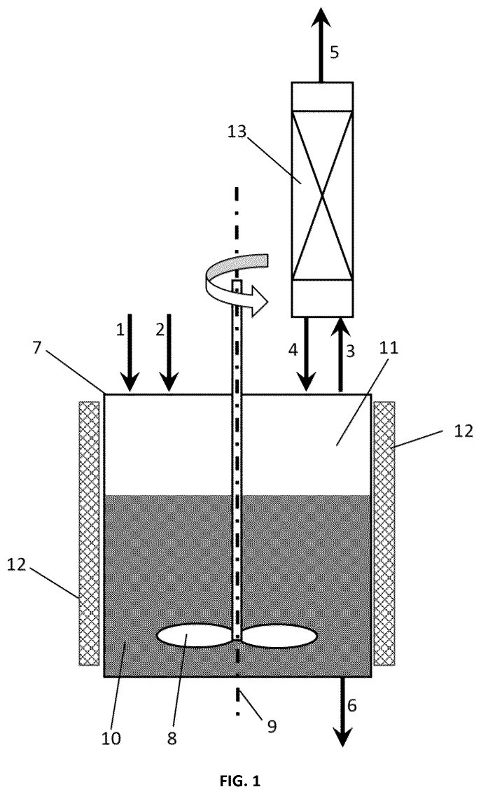

FIG. 1: shows a stirred tank reactor;

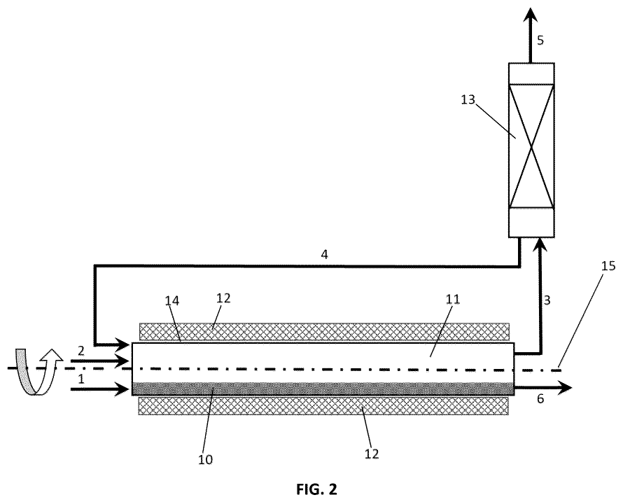

FIG. 2: a rotary tube reactor;

and

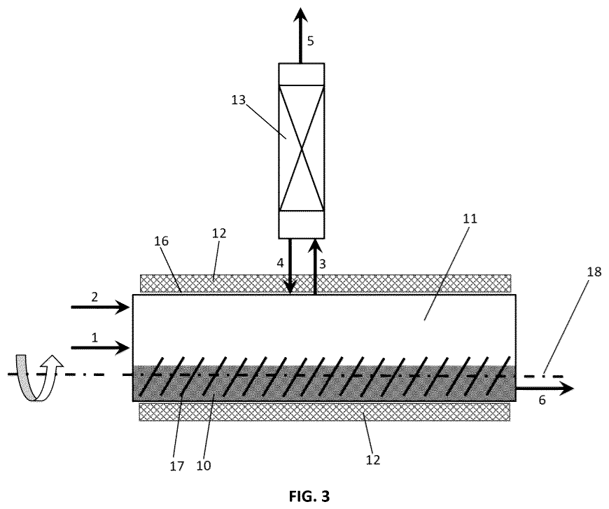

FIG. 3: a trough reactor (=conveying trough reactor).

There will firstly follow a brief summary of various possible embodiments of the invention:

In a first embodiment of the invention, which can be combined with all other embodiments, the conversion of the aminobenzoic acid is conducted at a reaction pressure (measured in the second phase) in the range from 0.10 bar to 10 bar, preferably 0.50 bar to 5.0 bar and particularly preferably 0.90 bar to 1.1 bar (especially ambient pressure).

In a second embodiment of the invention, which can be combined with all other embodiments, provided they are not restricted to a discontinuous process regime, over the duration of the conversion of the aminobenzoic acid in step (B), the aminobenzoic acid is continuously fed to the reactor and aniline and carbon dioxide are continuously discharged from the reactor.

In a third embodiment of the invention, which is a particular configuration of the second embodiment, the reactor is a stirred tank reactor having an inlet for the aminobenzoic acid, an outlet for the gaseous stream and an outlet for a liquid stream possibly containing solid particles (the conversion of the aminobenzoic acid is therefore effected [very substantially] with back-mixing).

In a fourth embodiment of the invention, which is a particular configuration of the third embodiment, the first phase comprises a proportion by mass of aniline, based on the total mass of aminobenzoic acid and aniline, in the range from 5.0% to 90%, preferably 10% to 70%.

In a fifth embodiment of the invention, which is another particular configuration of the second embodiment, the reactor comprises a reaction tube having an inlet for the aminobenzoic acid, an outlet for the gaseous stream and an outlet for a liquid stream possibly containing solid particles (the conversion of the aminobenzoic acid is therefore effected [very substantially] without back-mixing).

In a sixth embodiment of the invention, which is a particular configuration of the fifth embodiment,
the reaction tube is rotated about its longitudinal axis (it is then what is known as a rotary tube reactor, where the reaction tube may be inclined with respect to the horizontal and can optionally contain internals such as lifting strips, flow restrictors or a conveying screw)
or
the reaction tube is disposed in a fixed manner, with a conveying screw, which does not completely fill the cross section of the reaction tube, rotating in the reaction tube (this is then what is known as a trough reactor or conveying trough reactor).

In a seventh embodiment of the invention, which is a particular configuration of the fifth and sixth embodiments, a mixture containing 0.1% by mass to 30% by mass of aniline, preferably 1.0% by mass to 7.5% by mass, based on the total mass of the mixture, is continuously withdrawn from the reactor via the outlet for the liquid stream possibly containing solid particles.

In an eighth embodiment of the invention, which is a particular configuration of the second to seventh embodiments, an average residence time t_{V}, from the entry of an aminobenzoic acid molecule into the reactor to the discharge, via the gaseous stream, from the reactor of an aniline molecule formed therefrom, of 1.00 min to 500 min, preferably 5.00 min to 120 min, is set.

In a ninth embodiment of the invention, which can be combined with all other embodiments, provided they are not restricted to a continuous process regime, the aminobenzoic acid is converted discontinuously in batches, with the aminobenzoic acid being initially charged in the reactor and/or added to the reactor (continuously or at intervals) over a period t_{Z} and, after the entirety of a batch of aminobenzoic acid has been added, the latter is converted in the reactor at the reaction temperature and the reaction pressure for a (post-)reaction duration t_{R}.

In a tenth embodiment of the invention, which is a particular configuration of the ninth embodiment, the reactor is a stirred tank reactor.

In an eleventh embodiment of the invention, which is a particular configuration of the ninth and tenth embodiments, at the start of the (post-)reaction duration t_{R} the first phase comprises a proportion by mass of aniline, based on the total mass of aminobenzoic acid and aniline, in the range from 0.1% to 90%, preferably 5.0% to 90%, particularly preferably 10% to 70%.

In a twelfth embodiment of the invention, which is a particular configuration of the ninth to eleventh embodiments, 50% to 100% of the total amount of a batch of aminobenzoic acid is added (continuously or at intervals) to the reactor over the period t_{Z}, where t_{Z} is 30% to 70% of the total duration t_{Z}+t_{R}.

In a thirteenth embodiment of the invention, which is a particular configuration of the ninth to twelfth embodiments, the (post-)reaction duration t_{R} is in the range from 1.00 min to 500 min, preferably 5.00 min to 120 min.

In a fourteenth embodiment of the invention, which can be combined with all other embodiments, the gaseous stream containing aniline and carbon dioxide in step (B) passes through a condenser for the condensation of any aminobenzoic acid entrained in the gaseous stream (so that any entrained aminobenzoic acid is predominantly, preferably to an extent of at least 90%, condensed and then returned into the first phase located in the reactor, while aniline predominantly, preferably to an extent of at least 90%, passes through the condenser in gaseous form—in particular when there is also a (distillation or rectification) column arranged between the reactor and condenser).

In a fifteenth embodiment of the invention, ortho-aminobenzoic acid is provided in step (A). This embodiment can be combined with all other embodiments, provided that they do not provide for the provision of another aminobenzoic acid isomer in step (A).

In a sixteenth embodiment of the invention, para-aminobenzoic acid is provided in step (A). This embodiment can be combined with all other embodiments, provided that they do not provide for the provision of another aminobenzoic acid isomer in step (A).

In a seventeenth embodiment of the invention, which is a particular configuration of the fifteenth embodiment, step (A) comprises (i) the reaction of phthalimide or phthalic monoamide with an alkali metal hypohalide (especially sodium hypochlorite) in a basic medium followed by an acid treatment or (ii) the hydrogenation of 2-nitrobenzoic acid.

In an eighteenth embodiment of the invention, which is a particular configuration of the sixteenth embodiment, step (A) comprises (i) the reaction of terephthalic monoamide with an alkali metal hypohalide (especially sodium hypochlorite) in a basic medium followed by an acid treatment or (ii) the hydrogenation of 4-nitrobenzoic acid.

In a nineteenth embodiment of the invention, which is a particular configuration of the first to sixteenth embodiments, step (A) comprises the fermentation of a raw material containing a fermentable carbon-containing compound and a nitrogen-containing compound in the presence of microorganisms.

In a twentieth embodiment of the invention, which is a particular configuration of the nineteenth embodiment,
the fermentable carbon-containing compound is selected from starch hydrolyzate, sugarcane juice, sugarbeet juice, hydrolyzates of lignocellulosic raw materials or a mixture of two or more of the aforementioned carbon-containing compounds,
and
the nitrogen-containing compound is selected from ammonia gas, aqueous ammonia, (at least) one ammonium salt, soya protein, urea or a mixture of two or more of the aforementioned nitrogen-containing compounds.

In a twenty-first embodiment of the invention, which is a particular configuration of the nineteenth and twentieth embodiments, the microorganisms are selected from Escherichia coli, Pseudomonas putida, Corynebacterium glutamicum, Ashbya gossypii, Pichia pastoris, Hansenula polymorpha, Kluyveromyces marxianus, Yarrowia lipolytica, Zygosaccharomyces bailii or Saccharomyces cerevisiae.

In a twenty-second embodiment of the invention, which is a particular configuration of the first to twenty-first embodiments, step (B) is conducted without the addition of a catalyst and without adding aniline.

In a twenty-third embodiment of the invention, which is a further particular configuration of the first to twenty-first embodiments, step (B) is conducted in the presence of a catalyst selected from
(a) an aqueous mineral acid (such as in particular sulfuric acid, nitric acid and hydrochloric acid), (b) a zeolite (in particular of Y type in protonated form [H form]), (c) an Si—Ti molecular sieve, (d) a hydroxyapatite, (e) hydrotalcite, (f) an ion-exchange resin (in particular Amberlyst) and/or (g) an inorganic heterogeneous metal oxide catalyst containing a proportion by mass of Al₂O₃, based on the total mass of the metal oxides, of 40.0% to 100%, where the proportion by mass of Al₂O₃, based on the total mass of the inorganic heterogeneous metal oxide catalyst, is 25% to 100%.

In a twenty-fourth embodiment of the invention, which is a particular configuration of the twenty-third embodiment, the process comprises step (B)(iii), where the solvent comprises aniline (and in particular is aniline).

In a twenty-fifth embodiment of the invention, which is a further particular configuration of the first to twenty-first embodiments, the process comprises step (B)(iii), where the solvent comprises aniline (in particular is aniline) and no catalyst (extraneous to the system) is added.

In a twenty-sixth embodiment of the invention, which can be combined with all other embodiments (provided they do not provide for the use of aniline as solvent), in particular insofar as they relate to the use of a continuous-flow reaction tube in step (B), step (B)(iii) is included, where the solvent at the reaction temperature and reaction pressure remains in the first phase to an extent of at least 90%, preferably to an extent of at least 95%, particularly preferably to an extent of at least 99%.

In a twenty-seventh embodiment of the invention, which is a particular configuration of the twenty-sixth embodiment, the solvent is selected from a hydrocarbon (in particular having twelve or more carbon atoms), a silicone oil, an ether-based oil (in particular tri-, tetra- or pentaglyme), a molten salt, sulfolane, diphenyl ether, a haloaromatic (in particular trichlorobenzene) or an aniline-based amide (in particular 2-aminobenzanilide), with preference being given to a hydrocarbon or 2-aminobenzanilide.

In a twenty-eighth embodiment of the invention, which can be combined with all other embodiments, step (D) is conducted and comprises one of the following conversions:
(1) acid-catalyzed reaction of the aniline with formaldehyde to form di- and polyamines of the diphenylmethane series;
(2) acid-catalyzed reaction of the aniline with formaldehyde to form di- and polyamines of the diphenylmethane series, followed by reaction thereof with phosgene to form di- and polyisocyanates of the diphenylmethane series;
or
(3) conversion of the aniline to an azo compound.

The embodiments briefly outlined above and further possible configurations of the invention are elucidated in more detail hereinbelow. All embodiments and further configuration options are combinable as desired with one another unless the opposite is clearly apparent to a person skilled in the art from the context or an explicit statement is made otherwise.

In step (A) of the process according to the invention, aminobenzoic acid for conversion is provided. To this end, the aminobenzoic acid can be produced at the site of the decarboxylation or else be introduced from outside. In the process according to the invention, preference is given to using ortho- or para-aminobenzoic acid as starting material for the decarboxylation, with particular preference being given to ortho-aminobenzoic acid (anthranilic acid). Aminobenzoic acid can be prepared by any of the methods known in the prior art. Both chemical (non-fermentative) and biotechnological (fermentative) routes are known for the preparation of aminobenzoic acid. Either preparation type can be used in the context of the process according to the invention. Since these are well known and can all be applied without problems in the context of the decarboxylation according to the invention, they are outlined only briefly below:

Suitable chemical synthesis routes include in particular (i) a Hofmann rearrangement of the corresponding imides or monoamides with an alkali metal hypohalide (especially sodium hypochlorite) in a basic medium followed by an acid treatment and (ii) a hydrogenation of the corresponding nitrobenzoic acids. Starting materials in the first case (i) are therefore phthalimide or phthalic monoamide/terephthalic monoamide and in the latter case (ii) 2-nitrobenzoic acid or 4-nitrobenzoic acid.

Aminobenzoic acid can also be obtained via fermentative routes (cf. the patent literature cited at the outset). To this end, a raw material containing a fermentable carbon-containing compound and a nitrogen-containing compound is fermented in the presence of microorganisms. In this context, it is preferable that
the fermentable carbon-containing compound is selected from starch hydrolyzate, sugarcane juice, sugarbeet juice, hydrolyzates of lignocellulosic raw materials or a mixture of two or more of the aforementioned carbon-containing compounds,
and
the nitrogen-containing compound is selected from ammonia gas, aqueous ammonia, (at least) one ammonium salt, soya protein, urea or a mixture of two or more of the aforementioned nitrogen-containing compounds.

Suitable microorganisms include in particular Escherichia coli, Pseudomonas putida, Corynebacterium glutamicum, Ashbya gossypii, Pichia pastoris, Hansenula polymorpha, Kluyveromyces marxianus, Yarrowia lipolytica, Zygosaccharomyces bailii or Saccharomyces cerevisiae.

The fermentative route is preferred because it opens up a path toward a more sustainable and environmentally friendly production of aniline.

Step (B) of the process according to the invention relates to the actual decarboxylation of the aminobenzoic acid to give the desired product (or intermediate) aniline.

To this end, the aminobenzoic acid provided in step (A) is introduced (i) as a solid, (ii) in molten form or (iii) dissolved or suspended in a solvent into the decarboxylation reactor. If the aminobenzoic acid is introduced into the reactor in solid form (i), it melts there on account of the prevailing temperatures of 170° C. to 350° C., preferably 185° C. to 300° C., particularly preferably 190° C. to 260° C. In the case of melting (ii) the aminobenzoic acid outside of the decarboxylation reactor, it should in particular be ensured that the decarboxylation does not already start there. This can be ensured by the briefest possible residence times and the lowest possible temperatures. When using aniline as solvent (iii), that is to say when feeding aminobenzoic acid/aniline mixtures into the decarboxylation reactor (henceforth also referred to as reactor for short), a temperature of the solution of aminobenzoic acid in aniline prior to entry into the reactor of between room temperature and 160° C. should be chosen, depending on the mixing ratio. When the mixture enters the reactor it is heated to the reaction temperature, with the aniline added to the aminobenzoic acid passing to a very predominant extent into the second, gaseous, phase and being discharged from the reactor with the gaseous stream containing aniline and carbon dioxide. In addition, parallel to this the aminobenzoic acid is decarboxylated to further aniline, which also passes to a very predominant extent into the second, gaseous, phase and is discharged from the reactor with the gaseous stream containing aniline and carbon dioxide. When using solvents (iii) that are different from aniline, it should be ensured that these remain very substantially, and ideally completely, in the liquid phase, that is to say under the chosen reaction conditions have an appropriately high boiling point (which is generally the case when their boiling point at ambient pressure is >200° C.), and that, at the reaction temperature and the reaction pressure prevailing in the reactor, they in particular remain in the first phase (the liquid phase which possibly contains solid particles suspended therein) to an extent of at least 90%, preferably to an extent of at least 95%, particularly preferably to an extent of at least 99%. Suitable such solvents are high-boiling compounds that are unreactive toward the functional groups of the aniline and the aminobenzoic acid (amine and carboxyl group) under the chosen reaction conditions, such as long-chain hydrocarbons, in particular hydrocarbons having twelve or more carbon atoms (individual compounds or mixtures such as paraffin oil), silicone oils, ether-based oils (for example tri-, tetra- or pentaglyme), molten salts or high-boiling solvents such as sulfolane, diphenyl ether, halogenated aromatics (for example trichlorobenzene), 2-aminobenzanilide or other aniline-based amides. This embodiment is advantageous in particular when the reaction is conducted in continuous-flow reactors because the liquid discharge of the solvent enables an outlet (purge) for high-boiling impurities such as by-products and hence prevents the accumulation thereof in the reaction chamber.

The reaction pressure—with the proviso that an aniline-containing gas phase (the second phase) as described above forms—can be chosen within broad ranges and in particular is 0.10 bar to 10 bar, preferably 0.50 bar to 5.0 bar and particularly preferably 0.90 bar to 1.1 bar. In the context of the present invention, all reported pressures relate to absolute pressures. Very particularly preferably, the reaction can be conducted at ambient pressure, which is a particular advantage of the process according to the invention. To determine the pressure, a pressure measuring apparatus can be installed in the second (gaseous) phase. Measurement in the first phase is of course also possible, albeit not preferred. If the measured values deviate, the value measured in the second phase is decisive.

In a preferred embodiment, the gaseous stream withdrawn from the reactor and containing aniline and carbon dioxide passes through a condenser for the (selective) condensation of any aminobenzoic acid entrained in the gaseous stream. The condenser is operated in particular such that any entrained aminobenzoic acid is predominantly to completely, preferably to an extent of at least 90%, condensed, while aniline passes through the condenser (predominantly) in gaseous form. Condensed aminobenzoic acid is returned to the first phase (which is liquid and possibly contains solid particles) located in the reactor. In a preferred embodiment, the condenser is in the form of a plug-in condenser located directly above the reactor, so that the condensed aminobenzoic acid can flow freely out. To this end, use may be made of condenser types known per se in the specialist field. Particularly suitable designs feature the avoidance of stagnant liquid flow and can be mechanically cleaned. A secondary circuit of a cooling medium can be used to dissipate the heat. In a preferred execution, evaporative cooling with water is implemented in order thus to be able to utilize the waste heat for steam generation. When setting suitable operating conditions, the temperature should in particular be chosen to be sufficiently high in order to prevent crystallization of the aminobenzoic acid and hence avoid deposits. If necessary, the optimal operating conditions can be determined by simple preliminary experiments.

In order to minimize the proportion of aniline returned undesirably, a (distillation or rectification) column can be arranged between the reactor and the condenser. It is preferable that aniline passes through the condenser to an extent of at least 90%, preferably (essentially) completely, in gaseous form.

The decarboxylation can be conducted in the presence of a catalyst. Suitable catalysts (extraneous to the system) in particular include
(a) aqueous mineral acids (such as in particular sulfuric acid, nitric acid and hydrochloric acid), (b) zeolites (in particular of Y type in protonated form [H form]), (c) Si—Ti molecular sieves, (d) hydroxyapatites, (e) hydrotalcite, (f) ion-exchange resins (in particular Amberlyst) and/or (g) inorganic heterogeneous metal oxide catalysts containing a proportion by mass of Al₂O₃, based on the total mass of the metal oxides, of 40.0% to 100%, where the proportion by mass of Al₂O₃, based on the total mass of the inorganic heterogeneous metal oxide catalyst, is 25% to 100%.

Such a catalyzed reaction can be conducted with addition of aniline to the aminobenzoic acid to be converted (as described in WO 2018/002088 A1, where, however, purified aniline can also be used instead of crude aniline). However, it is also possible to conduct the decarboxylation with the addition of aniline to the aminobenzoic acid to be converted without using a catalyst (extraneous to the system) (as described in WO 2020/020919 A1). It is also possible to add neither aniline nor a catalyst (extraneous to the system).

The reaction regime can be discontinuous or continuous.

With a discontinuous reaction regime, the aniline is produced in discrete batches to which end corresponding batches of aminobenzoic acid are converted. The amount of aminobenzoic acid to be converted in the context of a batchwise production can be initially charged in the reactor or added (continuously or at intervals) thereto over a period t_{Z}. It is also possible to initially charge a portion of the aminobenzoic acid and to add the rest over the period t_{Z}. In any case, after adding the entire amount of a batch of aminobenzoic acid, the latter is converted in the reactor at the reaction temperature and reaction pressure for a (post-)reaction duration t_{R}. It is preferable to add 50% to 100% of the total amount of a batch of aminobenzoic acid continuously or at intervals to the reactor, with t_{Z} being 30% to 70% of the total duration of t_{Z}+t_{R}. For the (post-)reaction duration t_{R}, values of 1.00 min to 500 min, preferably 5.00 min to 120 min, are preferred. As reactors for conducting discontinuous aniline preparation, stirred tank reactors are suitable in particular.

At the start of the (post-)reaction duration t_{R} (that is to say after addition of the aminobenzoic acid has ended and the chosen conditions of reaction temperature and pressure have been reached), the first phase generally comprises a proportion by mass of aniline, based on the total mass of aminobenzoic acid and aniline, in the range from 0.1% to 90%, preferably 5.0% to 90%, particularly preferably 10% to 70%. The exact value depends, inter alia, on the nature of the aminobenzoic acid addition. If the aminobenzoic acid is initially charged at ambient temperature in the reactor and first melts in the reactor, of course first a small proportion of the aminobenzoic acid is converted when the reaction temperature is reached. If, on the other hand, the aminobenzoic acid is introduced, already in the molten state, into a reactor in which the desired conditions of reaction temperature and pressure already prevail, considerably more aminobenzoic acid will have already been converted when the addition is ended. Of course, a role is also played by how rapidly aniline that is formed passes from the first phase (which is liquid and possibly contains solid particles) into the second (gaseous) phase, which in turn depends essentially on the reaction temperature and pressure.

With a continuous reaction regime, the aniline is produced in continuous-flow reactors, where after a start-up phase a SteadyState is established in which a certain mass flow rate (for example reported in kg/h) of aminobenzoic acid is continuously supplied to the reactor and corresponding mass flow rates of aniline and carbon dioxide are continuously discharged from the reactor. Provided no operating disruptions occur, this steady state of the continuous conversion of aminobenzoic acid is maintained until the production is to be ended (for instance due to necessary maintenance work or quite simply because there is just no need for aniline). The reactor used must be equally well suited for possible highly concentrated suspensions (melting solids), low-viscosity liquids and possible high-viscosity liquids (by-products/residue).

A reactor type suitable for this purpose is a stirred tank reactor, which in contrast to the stirred tank reactor described further above in connection with the discontinuous process is operated such that, over the duration of the conversion of the aminobenzoic acid, aminobenzoic acid is continuously introduced into the reactor and the gaseous stream (containing aniline and carbon dioxide) is continuously withdrawn from the reactor. The stirred tank reactor must therefore have (at least) one inlet for the aminobenzoic acid and (at least) one outlet for the gaseous stream. Such a stirred tank reactor used in a continuous process should also have an outlet for a liquid stream, by means of which high-boiling by-products and other impurities possibly present can be discharged in liquid or suspended form. Recovery of reactant or product of value from this stream can be considered depending on the respective concentrations.

Such a stirred tank reactor is shown in FIG. 1. In this figure, the reference signs have the following meanings:

| | |
|---|---|
| 1 | Aminobenzoic acid input stream (solid, molten or |
| | dissolved/suspended) |
| 2 | Inert gas input stream (optional) |
| 3 | Exit for a (first) gaseous stream containing aniline, aminobenzoic |
| | acid, carbon dioxide, possibly water and possibly inert gas |
| 4 | Reflux for a liquid stream containing aminobenzoic acid |
| 5 | Exit for a (second) gaseous stream containing aniline and carbon |
| | dioxide, possibly water and possibly inert gas |
| 6 | Exit for a liquid (residue) stream, possibly containing suspended |
| | solid particles |
| 7 | Stirred tank |
| 8 | Stirrer |
| 9 | Axis of rotation of the stirrer |
| 10 | First phase (liquid, possibly containing solid particles suspended |
| | therein) |
| 11 | Gas space (second phase) |
| 12 | Heater |
| 13 | Condenser for the selective condensation of aminobenzoic acid |

When using a stirred tank reactor, the reaction takes place with substantial back-mixing, i.e. the composition of the reaction mixture, considered over the entire tank contents, is (essentially) constant, in contrast to the continuous-flow reaction tubes considered further below in which the composition of the reaction mixture continuously changes from the entry of the reactants to the exit of the products (or until the maximum conversion has been achieved). Preferably, in the process according to the invention, when using stirred tank reactors in a continuous process regime, the first phase comprises a proportion by mass of aniline, based on the total mass of aminobenzoic acid and aniline, in the range from 5.0% to 90%, preferably 10% to 70%. These numbers relate to a process in which aniline is not additionally also used as a solvent.

One possible configuration of the operation of such a stirred tank reactor is described in more detail hereinafter:

A stream of pulverulent or molten aminobenzoic acid is metered continuously into the stirred tank reactor. The stirred tank is heated; some time after reactant metering has begun a liquid level of molten aminobenzoic acid forms in the lower part thereof (establishment of the steady state). The aminobenzoic acid decomposes into aniline and carbon dioxide, with the temperature in the tank being chosen such that both products are gaseous and continuously leave the stirred tank reactor via an exit at the upper end thereof. A condenser is located at the gas exit and the operating temperature thereof is chosen such that entrained gaseous aminobenzoic acid is condensed as selectively as possible and flows back directly into the stirred tank reactor. The remaining gaseous stream arrives at a workup stage in which the target product aniline is separated from carbon dioxide, water that is generally present and any low-boiling impurities present. In the lower region of the stirred tank reactor, a small stream of the liquid vessel contents, possibly containing solid particles, is continuously drawn off, in order to avoid accumulation of high-boiling impurities in the stirred tank. This stream can optionally also be supplied to a workup stage in order to separate aminobenzoic acid present therein and aniline present therein from the residue. Depending on the composition of the products of value separated off, these are added either to the reactant stream of aminobenzoic acid or to the product stream of aniline (before or after workup thereof). The supply stream of pulverulent or molten aminobenzoic acid into the reactor must be chosen so that partial filling of the stirred tank with liquid results, that is to say be adapted to the required residence time for a complete reaction of the aminobenzoic acid and evaporation of the aniline formed. In the steady operating state of the continuously operated stirred tank reactor, the liquid level in the stirred tank should be constant. Optionally, a stream of inert gas (for example, preheated nitrogen) can be guided through the gas space of the stirred tank reactor in order to reduce the residence time of the gaseous product in the reactor.

Besides stirred tank reactors, tubular reactors (reaction tubes) are also suitable for conducting the process according to the invention. These have, at an end side or in the vicinity of an end side, an inlet for the aminobenzoic acid and downstream thereof in the upper region an outlet for the gaseous stream. They additionally have, as already mentioned for the stirred tank reactors, an outlet for a liquid stream (containing high-boiling by-products and any other impurities present) which is expediently disposed at the side opposite the inlet. In such reactors, the reaction proceeds very substantially without back-mixing, that is to say the composition of the reaction mixture flowing through the reactor continuously changes while passing through the reactor and at the point of exit of the liquid stream has the lowest concentration of aminobenzoic acid (=maximum conversion). Preference is given to conducting step (B) to the greatest possible extent without back-mixing. The liquid stream discharged via the outlet may contain solid particles, depending on the precise configuration of the reactor and reaction regime.

One suitable reactor type is the one known as a rotary tube reactor. This is a tubular reactor that during operation is rotated about its longitudinal axis. Such a rotary tube reactor is shown in FIG. 2. In this figure, the reference signs that have already been used in connection with FIG. 1 have the same meanings as they do there. The other reference signs have the following meanings:

| | |
|---|---|
| 14 | Rotary tube |
| 15 | Axis of rotation of the rotary tube |

The reactor can also—not shown in FIG. 2—be inclined with respect to the horizontal (i.e. in the direction of the flowing reaction mass) and optionally contain internals such as lifting strips, flow restrictors or a conveying screw.

One possible configuration of the operation of such a rotary tube reactor is described in more detail hereinafter:

A stream of pulverulent or molten aminobenzoic acid is metered continuously at an end side into a horizontal or inclined rotary tube reactor. The rotary tube reactor is heated, and the reactant metered in is conveyed through the tube depending on the rotary speed and inclination of the rotary tube. The aminobenzoic acid decomposes into aniline and carbon dioxide, with the temperature in the rotary tube being chosen such that both products are gaseous and continuously leave the reactor via an exit at the downstream end side of the rotary tube. In the process according to the invention, the rotary tube is partly filled; gaseous product formed during the reaction can thus flow past, above the material flowing at the bottom in the tube as a channel, to the gas exit. Located behind the gas exit is a condenser, the operating temperature of which is chosen such that any entrained gaseous aminobenzoic acid is as far as possible selectively condensed; this condensed stream is returned in suitable fashion to the entry of the rotary tube reactor. The remaining gaseous stream arrives at a workup stage in which the target product aniline is separated from carbon dioxide, water that is generally present and any low-boiling impurities present. In the channel of the material flowing at the bottom in the rotary tube, a gradient of the composition over the tube length arises in accordance with the reaction progress: While in the region of the entry the material consists of solid and/or molten aminobenzoic acid, at the exit side the liquid stream possibly containing solid particles will consist of high-boiling residue (which is possibly of high viscosity) and a small proportion of liquid aminobenzoic acid and aniline. This stream can be treated as described above for the stirred tank reactor. The supply stream of pulverulent or molten aminobenzoic acid to the rotary tube reactor must be chosen so that the proportion of product of value in the liquid residue is as low as possible, that is to say be adapted to the required residence time for a complete reaction of the aminobenzoic acid and evaporation of the aniline formed. The interior of the rotary tube can be empty or provided with internals, which for example in the form of blades can serve to improve transport, mixing and heat transfer or for example in the form of flow restrictors can contribute to increasing the amount of reaction mixture present in the rotary tube (what is called hold-up). Optionally, a stream of inert gas (for example, preheated nitrogen) can be added at the entry of the rotary tube reactor in order to reduce the residence time of the gaseous product in the reactor.

A further reactor type suitable for an essentially back-mixing-free reaction regime is the one known as a trough reactor (also called conveying trough reactor). This is a tubular reactor which, in contrast to the rotary tube reactor, is disposed in a fixed manner, where in the reactor a conveying screw rotates and does not completely fill the cross section of the reaction tube (the conveying screw fills only the lower region of the reactor; in the upper region only the gas phase is present). Such a trough reactor is shown in FIG. 3. In this figure, the reference signs that have already been used in connection with FIG. 1 have the same meanings as they do there. The other reference signs have the following meanings:

| | |
|---|---|
| 16 | Trough (also called conveying trough) |
| 17 | Conveying screw in the first phase |
| 18 | Axis of rotation of the conveying screw |

The trough reactor can—not shown in FIG. 3—be inclined with respect to the horizontal (i.e., in the direction of the flowing reaction mass). One possible configuration of the operation of such a trough reactor is described in more detail hereinafter: A stream of pulverulent or molten aminobenzoic acid is metered continuously at an end side into a horizontal or inclined trough reactor. The trough reactor is heated, and the product metered in is conveyed through the reactor depending on the rotary speed of the conveying screw and the inclination of the trough. The aminobenzoic acid decomposes into aniline and carbon dioxide, with the temperature in the trough reactor being chosen such that both products are gaseous and continuously leave the reactor via an exit at the upper side of the trough reactor. The trough reactor is partly filled so that gaseous product formed during the reaction can flow past, above the material flowing at the bottom in the trough in the region of the conveying screw as a channel, to the gas exit. A condenser is located at the gas exit and the temperature thereof is chosen such that any entrained gaseous aminobenzoic acid is condensed as selectively as possible and flows back directly into the trough reactor. The remaining gaseous stream arrives at a workup stage in which the target product aniline is separated from carbon dioxide, water that is generally present and any low-boiling impurities present. In the channel of the material conveyed by the screw at the bottom in the trough reactor, a gradient of the composition over the trough length arises in accordance with the reaction progress: While in the region of the entry the material consists of solid and/or molten aminobenzoic acid, at the exit side the liquid stream possibly containing solid particles will consist of high-boiling residue (which is possibly of high viscosity) and a small proportion of liquid aminobenzoic acid and aniline. This exit stream can be treated as described further above for the stirred tank reactor. The supply stream of pulverulent or molten aminobenzoic acid to the trough reactor must be chosen so that the proportion of product of value in the liquid residue is as low as possible, that is to say be adapted to the required residence time for a complete reaction of the aminobenzoic acid and evaporation of the aniline formed. The conveying screw of the trough reactor can have over its length conveying elements having different geometries in order to influence residence time behavior and mass transfer, for example different pitches or conveying directions of the screw elements. Optionally, a stream of inert gas (for example, preheated nitrogen) can be added at the entry of the trough reactor in order to reduce the residence time of the gaseous product in the reactor.

For all reactor types in an essentially back-mixing-free reaction regime, it is the case that a mixture containing 0.1% by mass to 30% by mass of aniline, preferably 1.0% by mass to 7.5% by mass, based on the total mass of the mixture, is preferably continuously withdrawn from the reactor via the outlet for the liquid stream possibly containing solid particles.

For all continuously operated process regimes (i.e., irrespective of whether or not operation with back-mixing takes place) of step (B), it is the case that the average residence time t_{V}, from the entry of an aminobenzoic acid molecule into the reactor to the discharge, via the gaseous stream, from the reactor of an aniline molecule formed therefrom, is 1.00 min to 500 min, preferably 5.00 min to 120 min.

Step (C) of the process according to the invention comprises the condensation and optional purification of the aniline withdrawn from the reactor in gaseous form. To this end, the gaseous stream withdrawn from the reactor, optionally after passing through a condenser (13 in the figures) for the removal of any entrained aminobenzoic acid by selective condensation, as described above, passes through a condenser (not shown in the figures) that is operated such that aniline is condensed out from the gaseous stream while carbon dioxide (possibly aside from small proportions that are present dissolved in the condensed aniline) passes through the condenser in gaseous form. To this end, use may be made of condenser types known per se in the specialist field. Particularly suitable designs feature the avoidance of stagnant liquid and can be mechanically cleaned. A secondary circuit of a cooling medium can be used to dissipate the heat. In a preferred execution, evaporative cooling with water is implemented in order thus to be able to utilize the waste heat for steam generation.

Here too, as already described above for the condenser 13 for selective condensation of any entrained aminobenzoic acid, a (distillation or rectification) column can be connected upstream of the condenser.

The aniline thus obtained already features a high purity, especially when a (distillation or rectification) column has been connected upstream of the condenser of step (C), as described above. If required, it can be further purified by distillation (especially heteroazeotropic distillation). Methods to achieve this are sufficiently well known in the specialist field and thus require no further explanation at this juncture.

Step (D) of the process according to the invention relates to the optional further conversion of the aniline obtained in (C). Possible further conversions include in particular the following:
(1) the acid-catalyzed reaction of the aniline with formaldehyde to form di- and polyamines of the diphenylmethane series;
(2) the acid-catalyzed reaction of the aniline with formaldehyde to form di- and polyamines of the diphenylmethane series, followed by reaction thereof with phosgene to form di- and polyisocyanates of the diphenylmethane series;
and
(3) the conversion of the aniline to an azo compound.

The further reaction of aniline with formaldehyde to give di- and polyamines of the diphenylmethane series (D)(1) is known per se and may be conducted by any prior art process. The continuous or partially discontinuous preparation of di- and polyamines of the diphenylmethane series from aniline and formaldehyde is disclosed, for example, in EP 1 616 890 A1, U.S. Pat. No. 5,286,760, EP-A-451442 and WO-A-99/40059. The reaction is effected under acid catalysis. A suitable acidic catalyst is preferably hydrochloric acid.

The further reaction of the thus obtained di- and polyamines of the diphenylmethane series with phosgene to give di- and polyisocyanates of the diphenylmethane series (D)(2) is also known per se and may be conducted by any prior art process. Suitable processes are described, for example, in EP 2 077 150 B1, EP 1 616 857 A1, EP 1 873 142 A1, and EP 0 314 985 B1.

The conversion of the aniline obtained according to the invention to azo compounds, in particular to azo dyes (D)(3) may also be effected by any prior art process. By way of example, reference may be made to the known preparation of Aniline Yellow (para-aminoazobenzene; CAS 493-5-7) or indigo (2,2′-bis(2,3-dihydro-3-oxomethylidene); CAS 482-89-3).

The invention is elucidated in more detail with reference to the examples which follow.

### EXAMPLES

### Compounds Used:

Anthranilic acid (AA, petrochemical): C₇H₇NO₂, purity >98%, Acros Organics
Anthranilic acid (AA, biogenic): C₇H₇NO₂, purity: 98%, Covestro Deutschland AG
2-Aminobenzanilide (AMD): C₁₃H₁₂N₂O, purity 95%, abcr GmbH

### Method Description:

HPLC: For HPLC analysis, a set-up from Agilent with UV detection (DAD, measured at 254 nm) was used. For separation, a column from Agilent (Eclipse XDB-C18; 5 μm; 4.6×150 mm) was used. The mobile phase used was a mixture of MeOH and buffer (MeOH:buffer volume ratio=40:60, buffer: 0.7 ml of 85% p.a. H₃PO₄ is diluted to a final volume of 11 with HPLC water, where the pH of 3.0 is to be set with aqueous sodium hydroxide solution before final filling). The flow rate was 0.7 ml/min. The temperature of the column oven was adjusted to 40° C. The injection volume was 0.5 μl. The retention times of the individual components aniline (ANL), anthranilic acid (AA) and 2-aminobenzanilide (AMD) were: ANL=2.6 min; AA=5.2 min; amide=14.9 min.

The peak areas are converted to area percent (area %). The quantification of the individual components in percent by mass (mass %), based on the reaction mixture, was enabled by calibration with pure substances beforehand. In addition to the mass composition, the values are used to determine the conversion of anthranilic acid, the yield of aniline formed, the selectivity of the aniline formation and the selectivity for the formation of 2-aminobenzanilide.

General Procedure (GP) 1: Decarboxylation of Anthranilic Acid with Complete Reflux of Aniline Formed (Comparison)

The set-up comprises an 80 ml four-necked flask with stirrer bar, a fitted jacketed dropping funnel, a reflex condenser and an inert gas connection (Ar). First, the dropping funnel is provided with the desired amount of solid AA at room temperature and the entire apparatus is inertized with a constant Ar stream of 20 standard I/h. The apparatus is operated open at ambient pressure. The reflux condenser is cooled to 5° C. After inertizing, the thermostat of the dropping funnel is set to 155° C. and a waiting period of 25 min is observed, until the AA has completely melted. At the same time, the reaction vessel is heated to 210° C. and transitions (reaction vessel-dropping funnel; reaction vessel-reflux condenser) are insulated. The AA is then added to the reaction vessel continuously over a defined period (t_{Z}). After the continuous metering, stirring is continued fora defined period (t_{R}) and then the system is run down. The product mass of the reaction vessel is determined and characterized via HPLC analysis with respect to AA, ANL and AMD composition.

GP 2: Decarboxylation of Anthranilic Acid with Gaseous Discharge of the Aniline Formed (According to the Invention)

The set-up comprises an 80 ml four-necked flask with stirrer bar, a fitted jacketed dropping funnel, a distillation bridge with distillation vessel and an inert gas connection (Ar). First, the dropping funnel is provided with the desired amount of solid AA at room temperature and the entire apparatus is inertized with a constant Ar stream of 20 standard liters/h. The apparatus is operated open at ambient pressure. The distillation vessel is cooled to 5° C. After inertizing, the thermostat of the dropping funnel is set to 165° C. and a waiting period of 25 min is observed, until the AA has completely melted. At the same time, the reaction vessel is heated to 210° C. and transitions (reaction vessel-dropping funnel; reaction vessel-distillation bridge) are insulated or trace heated. The AA is then added to the reaction vessel continuously over a defined period (t_{Z}). After the continuous metering, stirring is continued for a defined period (t_{R}) and then the system is run down. The product masses of the reaction and product vessel are determined and the fractions characterized via HPLC analysis with respect to AA, ANL and AMD composition.

The experiments are summarized in the tables below. The following abbreviations are used therein:
C=Comparative example
ANL=Aniline
AA=Anthranilic acid
AMD=2-Aminobenzanilide

TABLE 1
Decarboxylation of anthranilic acid to aniline. Reaction temperature T =
210° C.; inert gas stream (Ar) 20 Nl/h, 45 g anthranilic acid.

| | | | ω_{react} | ω_{dist} | ω(ANL_{react}) | ω(AA_{react}) | ω(AMD_{react}) | ω(ANL_{dist}) | ω(AA_{dist}) | ω(AMD_{dist}) |
|---|---|---|---|---|---|---|---|---|---|---|
| | t_{z}/ | tₜₒₜ/ | t = tₜₒₜ | t = tₜₒₜ | t = tₜₒₜ | t = tₜₒₜ | t = tₜₒₜ | t = tₜₒₜ | t = tₜₒₜ | t = tₜₒₜ |
| Ex. | min | min | [a] | [a] | [b] | [b] | [b] | [b] | [b] | [b] |
| 1 (GP 1) | 70 | 180 | 100 | — | 86.8 | 10 | 3.2 | — | — | — |
| 2 (GP 1) | 70 | 1560 | 100 | — | 78.8 | 1.57 | 19.6 | — | — | — |
| 3 (GP 2) | 70 | 180 | 4.0 | 96 | 0 | 0.18 | 3.6 | 95.1 | 1.1 | 0 |

| | | | Conversion_{AA}/ | Yield_{ANL}/ | Selectivity_{ANL}/ | Selectivity_{AMD}/ | Yield_{ANL, dist}/ | ANL purity/ | Final mass/ |
|---|---|---|---|---|---|---|---|---|---|
| | t_{z}/ | tₜₒₜ/ | % t = tₜₒₜ | % t = tₜₒₜ | % t = tₜₒₜ | % t = tₜₒₜ | % t = tₜₒₜ | % t = tₜₒₜ | g t = tₜₒₜ |
| Ex. | min | min | [c] | [d] | [e] | [e] | [f] | [g] | [h] |
| 1 (GP 1) | 70 | 180 | 93.0 | 90.0 | 96.8 | 3.18 | — | 86.8 | 30.2 |
| 2 (GP 1) | 70 | 1560 | 98.9 | 81.2 | 82.1 | 17.9 | — | 78.8 | 27.5 |
| 3 (GP 2) | 70 | 180 | 99.1 | 95.9 | 96.8 | 3.19 | 95.9 | 98.8 | 29.9 |

Explanatory notes for the tables:
[a] Mass distribution (figures are mass %) of the reaction mixture in reaction or distillation vessel at t = tₜₒₜ, with tₜₒₜ = t_{z} + t_{R};
[b] Proportion by mass in % in the product mixture, based on the total mass of the products in reaction and distillation vessel;
[c] Chemical conversion of anthranilic acid;
[d] Chemical yield of aniline;
[e] Selectivity for aniline or 2-aminobenzanilide;
[f] Chemical yield of aniline in % in the distillation vessel;
[g] Purity of the aniline (determined from the mass distribution of ANL, AA and AMD at t = t_{R} in the vessel considered);
[h] Final mass from the reaction vessel or sum of reaction and distillation vessel.

As shown by the examples, the process according to the invention enables the conversion of aminobenzoic acid with high conversions and low by-product formation, leading to a high yield of aniline. The process according to the invention exhibits a number of advantages over the process without distillative product separation. For the same reaction time, a higher conversion of AA and hence a higher yield of ANL can be seen in the case of a continuous distillative product take-off. For identical conversions of AA, an increased ANL selectivity and hence higher ANL yield can be observed in the case of a continuous distillative product take-off. An additional advantage is that the ANL in the case of the process according to the invention is obtained already in a high purity and hence there is less of a tendency in following process steps toward post-reactions or consecutive losses in yield.

1. A process for preparing aniline or an aniline conversion product, comprising:
(A) providing aminobenzoic acid;
(B) decarboxylating the aminobenzoic acid in a reactor,
wherein the aminobenzoic acid is introduced into the reactor (i) as a solid, (ii) in molten form or (iii) dissolved or suspended in a solvent and converted at a reaction temperature of 170° C. to 350° C. to aniline and carbon dioxide, wherein the conversion is conducted at a reaction pressure at which the boiling point of aniline is reached or exceeded, such that (a) first, liquid, phase possibly containing solid particles and (b) a second, gaseous, phase form in the reactor, with a gaseous stream containing aniline and carbon dioxide being discharged from the reactor; and
(C) condensing and optionally purifying the aniline present in the gaseous stream; and
(D) optionally, converting the aniline obtained in (C) to an aniline conversion product.

2. The process as claimed in claim 1, in which the conversion of the aminobenzoic acid is conducted at a reaction pressure of 0.10 bar to 10 bar.

3. The process as claimed in claim 1, in which, over the duration of the conversion of the aminobenzoic acid in step (B), the aminobenzoic acid is continuously fed to the reactor and aniline and carbon dioxide are continuously discharged from the reactor.

4. The process as claimed in claim 3, in which the reactor is a stirred tank reactor having an inlet for the aminobenzoic acid, an outlet for the gaseous stream and an outlet for a liquid stream possibly containing solid particles.

5. The process as claimed in claim 3, in which the reactor comprises a reaction tube having an inlet for the aminobenzoic acid, an outlet for the gaseous stream and an outlet for a liquid stream possibly containing solid particles.

6. The process as claimed in claim 5, in which the reaction tube
is rotated about its longitudinal axis
or
is disposed in a fixed manner, with a conveying screw, which does not completely fill the cross section of the reaction tube, rotating in the reaction tube.

7. The process as claimed in claim 1, in which the aminobenzoic acid is converted discontinuously in batches, with the aminobenzoic acid being initially charged in the reactor and/or added to the reactor over a period t_{Z} and, after the entirety of a batch of aminobenzoic acid has been added, the entirety of the batch is converted in the reactor at the reaction temperature and the reaction pressure for a reaction duration t_{R}.

8. The process as claimed in claim 7, in which the reactor is a stirred tank reactor.

9. The process as claimed in claim 1, in which the gaseous stream containing aniline and carbon dioxide in step (B) passes through a condenser for the condensation of any aminobenzoic acid entrained in the gaseous stream.

10. The process as claimed in claim 1,
(α) in which ortho-aminobenzoic acid is provided in step (A)
or
(β) in which para-aminobenzoic acid is provided in step (A).

11. The process as claimed in claim 10, in which
(α) step (A) comprises (i) the reaction of phthalimide or phthalic monoamide with an alkali metal hypohalide in a basic medium followed by an acid treatment or (ii) the hydrogenation of 2-nitrobenzoic acid,
or in which
(β) step (A) comprises (i) the reaction of terephthalic monoamide with an alkali metal hypohalide in a basic medium followed by an acid treatment or (ii) the hydrogenation of 4-nitrobenzoic acid.

12. The process as claimed in claim 1, in which step (A) comprises the fermentation of a raw material containing a fermentable carbon-containing compound and a nitrogen-containing compound in the presence of microorganisms.

13. The process as claimed in claim 1, in which
(α) step (B) is conducted without the addition of a catalyst and without adding aniline;
or in which
(β) step (B) is conducted in the presence of a catalyst comprising (a) an aqueous mineral acid, (b) a zeolite, (c) an Si—Ti molecular sieve, (d) a hydroxyapatite, (e) hydrotalcite, (f) an ion-exchange resin and/or (g) an inorganic heterogeneous metal oxide catalyst containing a proportion by mass of Al₂O₃, based on the total mass of the metal oxides, of 40.0% to 100%, where the proportion by mass of Al₂O₃, based on the total mass of the inorganic heterogeneous metal oxide catalyst, is 25% to 100%;
or in which
(γ) step (B)(iii) is included, where the solvent comprises aniline and no catalyst is added.

14. The process as claimed in claim 1, comprising step (B)(iii), in which the solvent at the reaction temperature and reaction pressure remains in the first phase to an extent of at least 90%, wherein the solvent is not aniline.

15. The process as claimed in claim 1, in which step (D) is conducted and comprises:
(1) acid-catalyzed reaction of the aniline with formaldehyde to form di- and polyamines of the diphenylmethane series;
(2) acid-catalyzed reaction of the aniline with formaldehyde to form di- and polyamines of the diphenylmethane series, followed by reaction thereof with phosgene to form di- and polyisocyanates of the diphenylmethane series;
or
(3) conversion of the aniline to an azo compound.
